(19) United States
(12) Patent Application Publication
(10) Pub. No.: US 2014/0187843 A1
(43) Pub. Date: Jul. 3, 2014

(54) RADIOISOTOPE-PHOTODYNAMIC THERAPY FOR CANCER TREATMENT

(71) Applicant: Joseph Friedberg, Philadelphia, PA (US)

(72) Inventor: Joseph Friedberg, Philadelphia, PA (US)

(21) Appl. No.: 14/112,991

(22) PCT Filed: Apr. 20, 2012
(86) PCT No.: PCT/US12/34517

§ 371 Date: Feb. 27, 2014

Related U.S. Application Data
(60) Provisional application No. 61/477,531, filed on Apr. 20, 2011.

(51) Int. Cl. A61N 5/06; A61N 5/10

(52) U.S. Cl. CPC A61N 5/062; A61N 5/10
USPC 600/1; 604/20

(57) ABSTRACT

The present invention provides compositions and methods for radioisotope-photodynamic therapy for treating cancer. Specifically, the invention relates to compositions and methods for administering a radiation emitter, a rare-earth doped down-converting nanophosphor and a photosensitizer for treating cancer.

### CROSS REFERENCE TO RELATED APPLICATIONS

This application claims priority to U.S. Provisional Patent Application 61/477,531, filed Apr. 20, 2011, which is incorporated by reference herein in its entirety.

### FIELD OF THE INVENTION

The invention relates to compositions and methods for radioisotope-photodynamic therapy for treating cancer. Specifically, the invention relates to compositions and methods for administering a radiation emitter, a down-converting nanophosphor, and a photosensitizer for treating cancer.

### BACKGROUND OF THE INVENTION

Cancers that involve surfaces of the pleura, pericardium and peritoneum are lethal malignancies that normally portend a life expectancy of several months. Although palliative chemotherapy may be attempted, for the vast majority of these cancers, there are no effective treatment options. Similarly metastatic cancers are, by definition, advanced stage and typically only treatable with palliative chemotherapy. Both situations are in need of novel therapeutic approaches.

One treatment that has been employed, with some reported success, has been the instillation of a radioisotope into the chest or abdominal cavity.

Photodynamic therapy (PDT) is a light based cancer treatment that requires visible light to activate a photosensitizing drug. The activated photosensitizer then transfers energy to oxygen, the excited species of which are thought to trigger a number of tumoricidal reactions. These mechanisms include direct cell kill, destruction of tumor neovasculature and, especially, stimulation of a tumor directed immune response. In clinical trials, researchers have used photodynamic therapy, in combination with surgery, to treat cancers of the pleura. A major limitation of PDT is that current photosensitizers, like common photovoltaic cells, are only able to absorb light in the visible spectrum. Visible light only penetrates tissue for one to several millimeters. To treat cancers of the pleura, for instance, surgeons have to open the patients chest just to deliver the light to the affected surfaces.

Metastatic disease, gross or microscopic, remains the most difficult form of cancer to treat due to the inumerable sites and locations and ability to defy any localized type of treatment. Current targeted systemic therapies are an addition to standard cytotoxic chemotherapies, but few cancers are suitable targets for these treatments.

Accordingly, there exists a need for compositions and methods for treating various forms of cancer.

### SUMMARY OF THE INVENTION

In one aspect, methods are provided for treating a cancer in a subject, the methods comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity. In an exemplary embodiment, said radiation emitter is an instillable radiation source and said down-converting nanophosphor is a rare-earth doped down-converting nanophosphor.

In another aspect, methods are provided for photodynamic therapy, the methods comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity.

In yet another aspect, methods are provided for radiation therapy, the methods comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity.

In a further aspect, methods are provided for inducing immune response against a cancer, the methods comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species.

In an other aspect, compositions are provided, the compositions comprising: a therapeutically effective amount of a radiation emitter; a down-converting nanophosphor that is capable of converting the radiation emitted from said radiation emitter into visible light; and a photosensitizer that is capable of being excited by said visible light to produce a reactive species.

In yet a further aspect, kits are provided, the kits comprising: a therapeutically effective amount of a radiation emitter; a down-converting nanophosphor that is capable of converting the radiation emitted from said radiation emitter into visible light; and a photosensitizer that is capable of being excited by said visible light to produce a reactive species.

Other features and advantages of the present invention will become apparent from the following detailed description examples and figures. It should be understood, however, that the detailed description and the specific examples, while indicating preferred embodiments of the invention, are given by way of illustration only, since various changes and modifications within the spirit and scope of the invention will become apparent to those skilled in the art from this detailed description. It is also contemplated that whenever appropriate, any embodiment of the present invention can be combined with one or more other embodiments of the present invention, even though the embodiments are described under different aspects of the present invention.

### DETAILED DESCRIPTION OF THE INVENTION

Disclosed herein are compositions and methods for radioisotope-photodynamic therapy for treating cancer. Specifically, embodiments of the invention are directed to compositions and methods for administering a radiation emitter, a down-converting nanophosphor and a photosensitizer for treating cancer.

In one aspect, provided herein are methods for treating a cancer in a subject, the methods comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity. Without wishing to be bound by theory, a reactive species may be, for example and without limitation, a reactive oxygen species such as a singlet oxygen or a free radical such as a superoxide free radical.

In another aspect, provided herein are methods for photodynamic therapy, the methods comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity. In yet another aspect, provided herein are methods for radiation therapy, the methods comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity. In a further aspect, provided herein are methods for inducing immune response against a cancer, the methods comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species.

In yet a further aspect, provided herein are compositions, the compositions comprising: a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity. In an other aspect, provided herein are kits, the kits comprising: a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity.

Rare earth phosphors are compounds created from rare earth elements that have the ability to absorb one type of energy and emit another type of energy. Down-converting phosphors produce visible light in response to radiation, and thus, according to certain embodiments, a rare-earth doped down-converting phosphor, a radiation emitter, and a photosensitizer are used to provide a radiation and/or photo-dynamic therapy (PDT).

In some embodiments, radiation therapy and PDT have an additive effect for treating cancer. In some embodiments, radiation therapy and PDT have a synergistic effect for treating cancer. In some embodiments, the combination therapies described herein are more effective relative to conventional therapies. In some embodiments, the combination therapies described herein are less toxic relative to conventional therapies and/or one of the corresponding monotherapies.

Any suitable radiation emitter (e.g., radioisotope) known to one of skilled in the art may be used. As known to one of skilled in the art, different radiation emitters may differ markedly in their properties, including, for example, the particular type or types of energy emitted therefrom, the mean and maximum energies of the emitted particles, the mean and maximum depths of penetration of the emitted particles in water or in other media, including, for example, soft biological tissue, and the like. In some embodiments, the radiation emitters may be included in a salt. The particular radiation emitter incorporated in the salts may affect the radioactive properties of the resulting radioactive salt The radiation emitter and the salt may be selected, as desired, based on the properties which are sought to be present in the radioactive salt.

Examples of a radiation emitter include, but are not limited to, a β-emitter, a α-emitter, a photon-emitter, ³²P, and tritium. In some embodiments, the radiation emitter is a β-emitter. In other embodiments, the radiation emitter is a α-emitter. In some embodiments, the radiation emitter is a photon-emitter. In some embodiments, the radioisotope is ³²P. In other embodiments, the radioisotope is a tritium. It will be appreciated that the selection of the radioisotope, the salt—if any, for a particular application will be within the discretion of a person skilled in the art.

In some embodiments, the radiation emitter of the invention is independently effective to treat a cancer. In other embodiments, the radiation emitters of embodiments of the present invention is effective in combination with other components as part of a greater scheme. For example, tritium is a radiation emitter that may have no direct effect on tissue, but may be effective to provide energy, for example to generate light via a converting phosphor, for exciting a photosensitizer so as to provide a photodynamic therapy.

The term “therapeutically effective amount”, as used herein, refers to an amount that provides a therapeutic effect for a given condition and administration regimen.

The term “treating”, as used herein, refers to any one or more of the following: delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics.

The term “substantially”, as used herein with respect to radioisotopes, refers to radioisotopes wherein the emitted particles are at least about 50%, preferably at least about 75%, and more preferably at least about 90%, radiation particles. Particularly preferred are radioisotopes having a mean energy of less than about 10 MeV, such as radioisotopes having a mean energy which ranges from about 0.1 to about 10 MeV, and all combinations and subcombinations of ranges therein. More preferably, the radioactive salts comprise radioisotopes having a mean energy of from about 0.3 to about 1.6 MeV, with radioisotopes having mean energies of from about 0.4 to about 1.4 MeV being even more preferred. Still more preferably, the radioactive salts comprise radioisotopes having a mean energy of from about 0.5 to about 1.2 MeV, with radioisotopes having mean energies of from about 0.6 to about 1 MeV being even more preferred. Particularly preferred are radioactive salts which comprise a radioisotope having a mean energy of from about 0.7 to about 0.8 MeV.

As noted above, certain radioisotopes of phosphorous may possess properties which make them especially useful in treatments.

The radioactive salts according to embodiments of the present invention may be especially useful in the treatment of cancer in a patient, although other patient treatments are also within the scope of the present invention. “Patient”, as used herein, refers to animals, including mammals, and preferably humans. In embodiments which involve the implantation of the radioisotope in a tumor in vivo may provide desirable exposure of the tumor to radiation while minimizing the exposure to radiation of nearby, normal tissue.

In certain preferred embodiments, the radioisotope is administered in combination with a pharmaceutically acceptable carrier. A wide variety of pharmaceutically acceptable carriers are available and can be combined with the present radioactive salts. Such carriers would be apparent to one skilled in the art, based on the present disclosure. Of course, any material used as a carrier is preferably biocompatible. Suitable carriers include, but are not limited to, water, buffer or saline solution. Other suitable carriers are described, for example, in Remington's, Pharmaceutical Sciences, Gennaro, A. R., ed., Mack Publishing Co., Easton, Pa. (1985), and The United States Pharmacopeia—The National Formulary, 22nd Revision, Mack Printing Company, Easton, Pa. (1990), the disclosures of each of which are hereby incorporated herein by reference, in their entirety.

The concentration of the radioisotope employed in the pharmaceutical compositions and/or the amount of radioisotope administered to the patient may vary and depends upon a variety of factors including, for example, the particular radioisotope and/or pharmaceutically acceptable carrier employed, the particular disease being treated, the extent of the disease, the size and weight of the patient, and the like. Typically, the radioisotope may be employed in the pharmaceutical compositions, and the compositions may be administered to a patient to provide initially lower levels of radiation dosages which may be increased until the desired therapeutic effect is achieved.

Any suitable nanophosphor may be used. In some embodiments, the nanophosphor is a down-converting nanophosphor. In some embodiments, the down-converting nanophosphor is a rare-earth doped down-converting nanophosphor. In some embodiments, the rare-earth doped down-converting nanophosphor is a lanthanide doped down-converting nanophosphor. For example, lanthanide ion doped down-converting phosphor nanocrystals (NCs) convert two or more photons of higher energy into one lower energy photon in the visible light range. In some embodiments, the nuclear powered radiation sources can be created as part of the crystal lattice itself (i.e., self-powered, self-glowing crystal). In other words, the radioisotope is incorporated into the crystal lattice of the phosphor nanocrystal. In one embodiment, the size of said nanophosphor range from about 0.5 nm to about 5000 nm.

In some embodiments, the rare-earth nanophosphor is synthesized using a single-step gas-phase flame synthesis method. In some embodiments, the phosphors are characterized by x-ray diffractometry, transmission electron microscopy, and fluorescence spectroscopy. In some embodiments, particle size, morphology, and photoluminescence intensity are affected by flame temperature. For example, gas-prepared nanophosphors are mostly single crystallites with an average size less than 30 nm.

In some embodiments, the rare-earth based crystal is about 1 nm to 50 nm. In some embodiments, the rare-earth based crystal is about 1 nm to 5 nm. In some embodiments, the rare-earth based crystal is about 3 nm to 6 nm. In some embodiments, the rare-earth based crystal is about 5 nm to 10 nm. In some embodiments, the rare-earth based crystal is about 8 nm to 12 nm. In some embodiments, the rare-earth based crystal is about 12 nm to 20 nm. In some embodiments, the rare-earth based crystal is about 15 nm to 25 nm. In some embodiments, the rare-earth based crystal is about 20 nm to 30 nm. In some embodiments, the rare-earth based crystal is about 30 nm to 40 nm. In some embodiments, the rare-earth based crystal is about 40 nm to 50 nm.

In other embodiments, the rare-earth based crystal is about 50 nm to 250 nm. In some embodiments, the rare-earth based crystal is about 40 nm to 80 nm. In some embodiments, the rare-earth based crystal is about 50 nm to 100 nm. In some embodiments, the rare-earth based crystal is about 80 nm to 120 nm. In some embodiments, the rare-earth based crystal is about 100 nm to 200 nm. In some embodiments, the rare-earth based crystal is about 170 nm to 250 nm.

In some embodiments, the size, shape (e.g., rod or sphere) and charge of the rare-earth nanophosphors can be used to target it to the cancer. For example, it is known that the permeability of the vasculature of cancerous tissue may be higher under certain conditions than that for the vasculature of non-cancerous tissue. Thus, in some embodiments, the rare-earth based crystals can be used that exploit this permeability differential to target the cancerous tissue by having a size, shape and charge that “leaks” through the vascularature of the cancerous tissue being treated but not of the vascularature of normal tissue.

In some embodiments, rare earth phosphors convert higher excitation wavelengths into a wide range of emission wavelengths in the visible spectrum. In other embodiments, rare earth phosphors compared to conventional fluorophores, have narrow emission bands, do not suffer from interference from autofluorescence or from photobleaching, and can be measured using relatively inexpensive detection equipment.

In some embodiments, efficient down-conversion luminescence, comprises selection of an efficient host material with less non-radioactive energy losses to accommodate lanthanide ions. For example, NaYF₄ matrixes, owing to its low vibrational energies and high ionicity, which lead to the minimum non-radiative quenching of the excited state of the rare earth ions, are used. NaYF₄ hosts of lanthanide NCs can occur in either alpha-phase (cubic) or Beta-phase (hexagonal) crystals. In preferred embodiment, Beta-phase (hexagonal) crystals are used as they exhibit 20-30 times higher upconverting efficiency than that of alpha-phase at similar crystal sizes.

In some embodiments, synthesis of colloidal NaYF₄ hosts doped with rare earth lanthanides by thermolysis of lanthanide and trifluoroacetic precursors in high boiling point solvents, including oleic acid (OA), oleylamine, and octadecene (ODE) is preformed. In some embodiments, the coordination ligands cap the NCs, and prevent them from agglomeration during crystal growth and nucleation at high temperature.

In some embodiments, the crystal or any of the components (e.g., the radioisotope) according to embodiments of the invention is labelled with a targeting agent. Targeting agents specifically targets a cell (e.g., a cancer cell) or a tissue (e.g., a tumor). Any suitable targeting agents, for example, a sugar, a protein, a fat, or a hormone can be used. In one example, as described more fully below, the targeting agent is a folate. In another example, the targeting agent is glucose or hyaluronic acid.

In some embodiments, targeting agents selectively bind to tumor tissue or cells versus normal tissue or cells of the same type. In certain embodiments the targeting agents are general ligands for cell surface receptors that are over-expressed in tumor tissue. Cell surface receptors over-expressed in cancer tissue versus normal tissue include, but are not limited to epidermal growth factor receptor (EGFR) (overexpressed in anaplastic thyroid cancer and breast and lung tumors), metastin receptor (overexpressed in papillary thyroid cancer), ErbB family receptor tyrosine kinases (overexpressed in a significant subset of breast cancers), human epidermal growth factor receptor-2 (Her2/neu) (overexpressed in breast cancers), tyrosine kinase-18-receptor (c-Kit) (overexpressed in sarcomatoid renal carcinomas), HGF receptor c-Met (overexpressed in esophageal adenocarcinoma), CXCR4 and CCR7 (overexpressed in breast cancer), endothelin-A receptor (overexpressed in prostate cancer), peroxisome proliferator activated receptor delta (PPAR-delta) (overexpressed in most colorectal cancer tumors), PDGFR A (overexpressed in ovarian carcinomas), BAG-I (overexpressed in various lung cancers), soluble type II TGF beta receptor (overexpressed in pancreatic cancer) folate and integrin (e.g.αvβ33). In some embodiments, the targeting agent is a cell surface receptor ligand for a receptor selected from the group consisting of folate, Her-2/neu, integrin, EGFR, metastin, ErbB, c-Kit, c-Met, CXR4, CCR7, endothelin-A, PPAR-delta, PDGFR A, BAG-I, and TGF beta.

In some embodiments, the targeting agent is an antibody or antigen-binding fragment that specifically binds to the desired target.

The term “antibody” refers herein to the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In each pair, the light and heavy chain variable regions (V_{L} and V_{H}) are together responsible for binding to an antigen, and the constant regions (C_{L}, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains V_{H}, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to V_{H}, Cγ1, Cγ2, Cγ3, V_{L}, and C_{L}.

Depending on the amino acid sequence of the constant domain of their heavy chains, intact antibodies can be assigned to different “classes.” There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The terms “antibody” or “antigen-binding fragment” respectively refer to intact molecules as well as functional fragments thereof, such as Fab, a scFv-Fc bivalent molecule, F(ab′)₂, and Fv that are capable of specifically interacting with a desired target. In some embodiments, the antigen-binding fragments comprise:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the is heavy chain; two Fab′ fragments are obtained per antibody molecule;

(3) (Fab′)₂, the fragment of the antibody that can be obtained by treating whole antibody with the enzyme pepsin without subsequent reduction; F(ab′)2 is a dimer of two Fab′ fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

(6) scFv-Fc, is produced in one embodiment, by fusing single-chain Fv (scFv) with a hinge region from an immunoglobulin (Ig) such as an IgG, and Fc regions.

In some embodiments, the antibody provided herein is a monoclonal antibody. In other embodiments, the antigen-binding fragment provided herein is a single chain Fv (scFv), a diabody, a tandem scFv, a scFv-Fc bivalent molecule, an Fab, Fab′, Fv, or F(ab═)₂.

The term “Bivalent molecule” or “BV” refers to a molecule capable of binding to two or more separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art.

In some embodiments, the crystal linked to the antibody or antigen-binding fragment as described herein is a 5-40 nm-diameter nanophosphor crystal. In some embodiments, the crystal linked to the antibody or antigen-binding fragment as described herein is a 10-30 nm-diameter nanophosphor crystal. In some embodiments, the crystal linked to the antibody or antigen-binding fragment as described herein is a 25-35 nm-diameter nanophosphor crystal. In some embodiments, the crystal linked to the antibody or antigen-binding fragment as described herein possesses narrower emission bands than type-II quantum dots (QDs). In some embodiments, the crystal linked to the antibody or antigen-binding fragment as described herein is a visible light nanophosphor crystal. In some embodiments, the nanophosphor crystal possesses an irradiance larger than an equivalent emission wavelength 30 nm quantum dot that bears an organic biocompatible coating and an appropriate antibody-based-targeting agent. In other embodiments, provided herein are compositions, the compositions comprising: antibody reagents labeled with rare-earth nanocrystal (NC) reporters.

In another aspect, provided herein are compositions: the compositions comprising a photodynamic reporter. In some embodiments, the photodynamic reporter is an antibody or antigen-binding fragment labeled with a rare-earth phosphor nanocrystal. In some embodiments, the photodynamic reporter is a rare-earth phosphor nanocrystal linked to an antibody or antigen-binding fragment. In some embodiments, the photodynamic reporter is a rare-earth phosphor nanocrystal attached to an antibody or antigen-binding fragment. In some embodiments, the photodynamic reporter is a rare-earth phosphor nanocrystal conjugated to an antibody or antigen-binding fragment. In some embodiments, the photodynamic reporter is a rare-earth phosphor nanocrystal chemically attached to an antibody or antigen-binding fragment.

In some embodiments, the nanophosphor composition comprises a stabilizer. For example, the stabilizer is a protein, such as Bovine Serum Albumin (BSA). In some embodiments, the composition comprises sodium azide at concentrations ranging from about 0.02 to about 0.05% (w/v). In some embodiments, the composition comprises glycerol.

In some embodiments, the nanophosphor composition comprises a polyol. In some embodiments, the composition comprises a buffer such as acetate (e.g., sodium acetate, potassium acetate, magnesium acetate) and acetic acid (e.g., at a concentration of about 1 mM to about 20 mM) and sucrose (e.g., at a concentration of about 5 mg/mL to about 70 mg/mL). In some embodiments, the composition is at a pH of about 4.5 to about 7.0. In some to embodiments, the composition is at a pH of about 5.5 to about 6.0.

In some embodiments, the composition is a stable aqueous formulation comprising an effective amount of an antibody or antigen-binding fragment. In another embodiment, the composition is a stable aqueous formulation comprising a an effective amount of an antibody or antigen-binding fragment not subjected to prior lyophilization.

Any suitable photosensitizer known to one of skilled in the art may be used. For efficient treatment, efficient photosensitizers for reactive species generation can be used. Many organic dyes, porphyrins and their derivatives, flavins, and organometallic species such as bis-cyclometallated Ir(III) complexes are known to be efficient photosensitizers (PSs) and can be used. In some embodiments, a photofyrin (e.g., a mixture of porphyrins, including photoporphyrin, haematoporphyrin, hydroxyethyldeuteropophyrin), a verteporfin, or a benzoporphyrin can also be used.

Fullerenes are also good candidates for PDT and medical applications. The efficient generation of reactive species by photoexcited C₆₀ and C₇₀ makes fullerenes useful for PDT. However, fullerenes absorb strongly in the UV and moderately in the visible region.

It has been demonstrated that nanostructured materials can be photoactivated to produce reactive species. In order to deliver the luminescent nanoparticles and the photosensitizers to the targeted tissue, preferably they should be packaged together. In addition, the package preferably should be compact in order to promote energy transfer from the nanoparticles to the photosensitizers thereby allowing efficient photoactivation to be accomplished. A typical mechanism for energy transfer is fluorescence resonance energy transfer (FRET). As used herein, FRET refers to the transfer from the initially excited donor (the scintillation nanoparticle) to an acceptor (the photosensitizer). A characteristic of FRET is that the transfer rate is highly dependent on the distance between the donor and receptor. The distance at which FRET is 50% efficient—called the Forster distance—is typically 2-10 nm. Generally, in order to have an efficient energy transfer, the distance between the donor and the acceptor should be less than 10 nm. This distance rule imposes limitations on the selection of the linkers and packaging options.

In some embodiments, two or more of a radiation emitter, a down-converting nanophosphor, and a photosensitizer are operably linked to each other. Any suitable linking method known to one of skilled in the art may be used. In some embodiments, two or more of a radiation emitter, a down-converting nanophosphor, and a photosensitizer are conjugated to each other. For example, a radiation emitter is conjugated to a down-converting nanophosphor. In another example, a down-converting nanophosphor is conjugated to a photosensitizer. In some embodiments, any permutation of a radiation emitter, a lanthanide converter, and a photosensitizer are conjugated to each other. For example, a radiation emitter is conjugated with a lanthanide converter and/or a photosensitizer. In some embodiments, a radiation emitter is conjugated to a lanthanide converter and the conjugated composition and a photosensitizer are administered independently. In some embodiments, a photosensitizer is conjugated to a lanthanide converter and the conjugated composition and a radiation emitter are administered independently. In some embodiments, a radiation emitter is conjugated to a photosensitizer and the conjugated composition and a lanthanide converter are administered independently.

the term “cancer” and “cancerous” refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies. Specific cancers treated according to certain embodiments of the present invention are cancers that involve surfaces of the pleura, pericardium and peritoneum, as well as metastatic cancers (i.e., where the cancer cells have spread to ectopic sites). Embodiments of the present invention, may be particularly useful in treating primary or secondary cancers of the chest, abdominal, spinal or pericardial spaces. For example, embodiments of the present invention, include but are not limited to the treatment of malignant peritonitis, malignant pleuritis, malignant meningitis, and/or malignant pericarditis.

There are separate mechanisms, for example, for radiation therapy, photodynamic therapy, immunotherapy, and chemotherapy by which a cancer can be treated. In some embodiments, these mechanisms can work in combinations. In other embodiments, these mechanisms can work individually, separately, or independently. It is known in the art that the treatment can be tunable such that different preparations could be prepared to favor one type of mechanism or to tailor the mechanism for the susceptibility of different cancers.

A photodynamic therapy effect may include, for example, apoptosis, direct cell kill, destruction of neovasculature, stimulation of immune effect. A radiation therapy effect may vary depending on the energy level and type of radiation (e.g., alpha, beta, or photon emission).

In a preferred embodiment, a cancer is treated according to methods provided herein by inducing an immunotherapeutic effect. Photodynamic therapy is one of the most effective ways to stimulate an immune effect. In some embodiments, low doses of photodynamic therapy over a prolonged period of time or metronomic photodynamic therapy can be used as a technique for stimulating an immunotherapeutic effect. In some embodiments, photodynamic therapy and radiation therapy can act synergistically. In some embodiments, photodynamic therapy and radiation therapy can act synergistically to stimulate an immune effect against a cancer.

In some embodiments, photodynamic therapy and radiation therapy is used in combination with an immunotherapy. For example, low doses of cyclophosphamide, BCG, or an interferon in combination with one or more other methods described herein are used to stimulate an immune effect against a cancer Immunotherapeutic approaches, also include but are not limited to, ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies. It will be appreciated that any appropriate immunotherapy know in the art may be used, and that the selection of a particular immunotherapy to achieve a desired therapeutic effect is within the discretion of a person skilled in the art.

Embodiments of the invention can be used to treat any disease or disorder where radiation therapy, photodynamic therapy, and/or immunotherapy can be useful. In some embodiments, the invention is directed to treating a cancer disease using radiation therapy, photodynamic therapy, and/or immunotherapy. In some embodiments, the invention is directed to treating a mesothelioma. In some embodiments, the invention is directed to treating a pleura associated cancer. In some embodiments, the invention is directed to treating a pericardium associated cancer. In some embodiments, the invention is directed to treating a peritoneum associated cancer. In some embodiments, the invention is directed to treating a pericardium where malignant effusions that are difficult to treat. In some embodiments, the invention is directed to treating a meningeal carcinomatosis. In some embodiments, the invention is directed to treating a metastatic cancer. Other lethal malignancies requiring radiation therapy, photodynamic therapy, immunotherapy and/or chemotherapy can also be treated according to certain embodiments of the invention.

In certain embodiments, the radiation therapy and photodynamic therapy is co-administered with one or more other therapeutic agents or treatments. Other therapeutically effective agents/treatments include surgery, anti-angiogenesis agents, antibodies to other targets, small molecules, photodynamic therapy, cytotoxic agents, cytokines, chemokines, growth inhibitory agents, anti-hormonal agents, kinase inhibitors, cardioprotectants, immunostimulatory agents, immunosuppressive agents, and agents that promote proliferation of hematological cells.

The compositions according to certain embodiments of the invention can be administered locally, systemically, or a combination thereof, by any suitable methods known to one of skilled in the art. In certain embodiments, a radiation emitter, a lanthanide converter, and a photosensitizer are administered by any permutation of locally, systemically, or combination thereof. For example, a radiation emitter is conjugated to a lanthanide converter which is administered locally and a photosensitizer is administered systemically, or vice-versa. In another example, a photosensitizer is conjugated to a lanthanide converter which is administered locally and a radiation emitter is administered systemically, or vice-versa. In a further example, a radiation emitter is conjugated to a photosensitizer which is administered locally and a lanthanide converter is administered systemically, or vice-versa.

In some embodiments, a composition described herein is administered locally, such as by implantation. In some embodiments, a composition described herein is administered by systemically. As appropriate, a composition described herein may be administered intraperitoneally, intrapleurally, intrapericardially or intrathecally and formulated in a form suitable for such administration, respectively. In other embodiments, a composition described herein is administered by intravenous, intraarterial, or intramuscular injection of a liquid preparation. Suitable liquid formulations include solutions, suspensions, dispersions, emulsions, oils and the like. In some embodiments, a composition described herein is administered intravenously and is thus formulated in a form suitable for intravenous administration. In some embodiments, a composition described herein is administered intraarterially and is thus formulated in a form suitable for intraarterial administration. In some embodiments, a composition described herein is administered intramuscularly and is thus formulated in a form suitable for intramuscular administration.

In some embodiments, the compositions described herein may be delivered to targets, such as cancerous lesions. In some embodiments, folate-conjugation is used for targeted delivery. Folates are low molecular weight pterin-based vitamins required by eukaryotic cells for one-carbon metabolism and de novo nucleotide synthesis. The folate receptor is a glycosylphosphatidylinositol-anchored, high-affinity membrane folate binding protein that is over expressed in a wide variety of human tumors, including more than 90% of ovarian carcinomas. On the other hand, normal tissue distribution of the folate receptor is highly restricted, making it a useful marker for targeted drug delivery to tumors. Folic acid, a high-affinity ligand for the folate receptor, retains its receptor binding property when covalently derivatized by its gamma-carboxyl group. Studies have shown that folate conjugates are taken into receptor-bearing tumor cells via folate receptor-mediated endocytosis. Folate-conjugation, therefore, presents a useful method for receptor-mediated drug delivery into receptor-positive tumor cells. In some embodiments, the compositions described herein are delivered in a vesicle, e.g., a liposome.

In some embodiments, carriers or diluents used in methods of the present invention include, but are not limited to, a gum, a starch (e.g., corn starch, pregeletanized starch), a sugar (e.g., lactose, mannitol, sucrose, dextrose), a cellulosic material (e.g. microcrystalline cellulose), an acrylate (e.g., polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In some embodiments, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In other embodiments, the compositions further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), antioxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., aspartame, citric acid), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In some embodiments, the compositions described herein are packaged in a way that makes it easily accessible or absorbed by a tumor. In some embodiments, the compositions described herein are packaged in a liposome, a micelle, or other suitable carriers known to one of skilled in the art.

In some embodiments, the compositions provided herein are controlled-release compositions, i.e. compositions in which the active compound is released over a period of time after administration. Controlled- or sustained-release compositions include formulation in lipophilic depots (e.g., fatty acids, waxes, oils). In other embodiments, the compositions provided herein are immediate-release compositions, i.e. compositions in which the active compound is released immediately after administration.

In other embodiments, the pharmaceutical composition is delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In other embodiments, polymeric materials are used; e.g. in microspheres in or an implant. In yet other embodiments, a controlled release system is placed in proximity to the target cell, thus requiring only a fraction of the systemic dose (see, e.g., Goodson, in Medical Applications of Controlled Release, supra, vol. 2, pp. 115-138 (1984); and Langer R, Science 249: 1527-1533 (1990).

The compositions also include, in other embodiments, incorporation of the active material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

Also encompassed by embodiments of the present invention are particulate compositions coated with polymers (e.g., poloxamers or poloxamines) and the compounds described herein coupled to antibodies or antigen-binding fragments directed against tissue-specific receptors, ligands or antigens or coupled to ligands of tissue-specific receptors.

Also encompassed by embodiments of the present invention are compounds described herein modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In some embodiments, the methods of the present invention further comprise administering one or more chemotherapeutic agents. It will be appreciated that any appropriate chemotherapeutic agents know in the art may be used, and that the selection of a particular chemotherapeutic agent to achieve a desired therapeutic effect is within the discretion of a person skilled in the art. Examples of chemotherapeutic agents include, but are not limited to, cisplatin, alkylating agents such as thiotepa and cyclosphosphamide (CYTOXAN); alkyl sulfonates such as busulfan, improsulfan and piposulfan; androgens such as calusterone, dromostanolone propionate, epitiostanol, mepitiostane, testolactone; anti-adrenals such as aminoglutethimide, mitotane, trilostane; anti-androgens such as flutamide, nilutamide, bicalutamide, leuprolide, and goserelin; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti estrogens including for example tamoxifen, raloxifene, aromatase inhibiting 4(5)-imidazoles, 4-hydroxytamoxifen, trioxifene, keoxifene, LY 117018, onapristone, and toremifene (Fareston); anti-metabolites such as methotrexate and 5-fluorouracil (5-FU); folic acid analogues such as denopterin, methotrexate, pteropterin, trimetrexate; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; folic acid replenisher such as frolinic acid; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; platinum analogs such as cisplatin and carboplatin; vinblastine; platinum; proteins such as arginine deiminase and asparaginase; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; taxanes, e.g. paclitaxel (TAXOL, Bristol-Myers Squibb Oncology, Princeton, N.J.) and docetaxel (TAXOTERE, Rhone-Poulenc Rorer, Antony, France); topoisomerase inhibitor RFS 2000; thymidylate synthase inhibitor (such as Tomudex); additional chemotherapeutics including aceglatone; aldophosphamide glycoside; aminolevulinic acid; amsacrine; bestrabucil; bisantrene; edatraxate; defofamine; demecolcine; diaziquone; difluoromethylornithine (DMFO); elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone;

2,2′,2″- trichlorotriethylamine; urethan; vindesine; dacarbazine; mannomustine; mitobronitol; mitolactol; pipobroman; gacytosine; arabinoside (“Ara-C”); cyclophosphamide; thiotepa; chlorambucil; gemcitabine; 6-thioguanine; mercaptopurine; methotrexate; etoposide (VP-16); ifosfamide; mitomycin C; mitoxantrone; vincristine; vinorelbine; navelbine; novantrone; teniposide; daunomycin; aminopterin; xeloda; ibandronate; CPT-11; retinoic acid; esperamicins; and capecitabine; and pharmaceutically acceptable salts, acids or derivatives of any of the above.

The methods described herein include using a combination therapy. The term “combination” is used in its broadest sense and means that a subject is treated with at least two therapeutic regimens. Treatment with photodynamic therapy in combination with a therapeutically radioactive isotope can be simultaneous (concurrent), consecutive (sequential, in either order), or a combination thereof. For example, a subject undergoing combination therapy receives treatment with both of these therapeutic agents at the same time (i.e., simultaneously) or at different times (i.e., sequentially, in either order, on the same day, or on different days), so long as the therapeutic effect of the combination of both substances is caused in the subject undergoing therapy. Where these two therapeutic agents are administered simultaneously, they can be administered as separate pharmaceutical compositions, or they can be administered as a single pharmaceutical composition comprising both of these therapeutic agents.

All sequence citations, accession numbers, references, patents, patent applications, scientific publications or other documents cited are hereby incorporated by reference.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### EXAMPLES

### Example 1

### Combination of Radiation and Photodynamic Therapy

Surprisingly and unexpectedly, a radioactive isotope can be used to generate visible light for the purpose of activating photosensitizers for photodynamic therapy to treat cancer.

An isotope, for example, a tritium, is instilled into the chest cavity, abdomen or pericardium, and have it serve as not only a radiation therapy treatment, but also as the energy source to intracorporeally generate visible light for photodynamic therapy.

A lanthanide based down-converting nanophosphor is used for photodynamic therapy. This phosphor is capable of converting the radiation emitted from the isotope into visible light. This phosphor is conjugated to a photosensitizer such as porphyrin which is capable of being excited by the visible light.

After administering the isotope, phosphor, and photosensitizer, a reactive species is produced. Such reactive species has a tumoricidal activity and kills the tumor cells.

Having described preferred embodiments of the invention with reference to the accompanying drawings, it is to be understood that the invention is not limited to the precise embodiments, and that various changes and modifications may be effected therein by those to skilled in the art without departing from the scope or spirit of the invention as defined in the appended claims.

1. A method for treating a cancer in a subject comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity.

2. The method of claim 1, wherein said radiation emitter is an α-emitter, a β-emitter, or a photon emitter.

3. The method of claim 1, wherein said radiation emitter is a tritium.

4. The method of claim 1, wherein said radiation emitter is a ³²P isotope.

5. The method of claim 1, wherein said down-converting nanophosphor is a rare-earth doped down-converting nanophosphor, and wherein said rare-earth is lanthanide.

6. The method of claim 1, wherein said nanophosphor comprises a crystal lattice and said radiation emitter is incorporated into said crystal lattice.

7. The method of claim 1, wherein the size of said nanophosphor ranges from about 0.5 nm to about 5000 nm.

8. The method of claim 1, wherein said visible light has a wavelength ranging from about 380 nm to 750 nm.

9. The method of claim 1, wherein said photosensitizer is a porphyrin.

10. The method of claim 1, wherein said cancer is a cancer of a pleura, a pericardium, a peritoneum, a malignant effusion, a menningeal carcinomatosis, a metastatic cancer.

11. (canceled)

12. (canceled)

13. The method of claim 1, wherein one or more of said radiation emitter, said phosphor, and said photosensitizer are locally administered.

14. The method of claim 1, wherein one or more of said radiation emitter, said phosphor, and said photo sensitizer are systemically administered.

15. The method of claim 1, wherein one or more of said radiation emitter, said phosphor, and said photosensitizer are operably linked.

16. The method of claim 1, wherein one or more of said radiation emitter, said phosphor, and said photosensitizer are operably linked by a conjugate or covalent bond.

17. The method of claim 1, wherein at least one of said radiation emitter, said phosphor, and said photosensitizer is operably linked to a target moiety specific to a tumor associated with said cancer.

18. The method of claim 17, wherein said target moiety is an antibody or an antigen-binding fragment.

19. A method for a photodynamic therapy comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity.

20.-23. (canceled)

24. The method of claim 19, wherein said down-converting nanophosphor is a rare-earth doped down-converting nanophosphor, and wherein said rare-earth is lanthanide.

25. The method of claim 19, wherein said nanophosphor comprises a crystal lattice and said radiation emitter is incorporated into said crystal lattice.

26.-30. (canceled)

31. A method for radiation therapy comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity.

32.-34. (canceled)

35. The method of claim 31, wherein said down-converting nanophosphor is a rare-earth doped down-converting nanophosphor, and wherein said rare-earth is lanthanide.

36. The method of claim 31, wherein said nanophosphor comprises a crystal lattice and said radiation emitter is incorporated into said crystal lattice.

37.-41. (canceled)

42. A method for inducing an immune response to treat a cancer, the method comprising: administering a therapeutically effective amount of a radiation emitter; a down-converting nanophosphor that is capable of converting the radiation emitted from said radiation emitter into visible light; and a photosensitizer that is capable of being excited by said visible light to produce a reactive species.

43.-53. (canceled)

54. A kit comprising: a therapeutically effective amount of a radiation emitter; a down-converting nanophosphor that is capable of converting the radiation emitted from said radiation emitter into visible light; and a photosensitizer that is capable of being excited by said visible light to produce a reactive species having a tumoricidal activity.
